(19) United States
(12) Patent Application Publication
(10) Pub. No.: US 2026/0240864 A1
(43) Pub. Date: Aug. 20, 2026

(54) Compositions, dose regimens and combinations of Nerandomilast in the treatment of IPF and PPF

(71) Applicant: Boehringer Ingelheim International GmbH, Ingelheim am Rhein (DE)

(72) Inventors: Christian Benedikt Otto HESSLINGER, Tettenweis (DE); Dilara JAPPAR, Herndon, VA (US); Yi LIU, Millwood, NY (US); Peter NICKOLAUS, Warthausen (DE); Susanne STOWASSER, Lorzweiler (DE); Florian VOSS, Mainz (DE); Yichao YU, Wilton, CT (US); Donald ZOZ, Lebanon, CT (US)

(21) Appl. No.: 19/539,861

(22) Filed: Feb. 13, 2026

Related U.S. Application Data
(60) Provisional application No. 63/758,698, filed on Feb. 14, 2025.

(30) Foreign Application Priority Data
Feb. 20, 2025 (EP) 25159108.7
May 15, 2025 (EP) 25176721.6

(51) Int. Cl. A61K 31/5377; A61K 9/00; A61K 9/20; A61K 31/437; A61K 31/44; A61K 31/443; A61K 31/444; A61K 31/519

(52) U.S. Cl. CPC A61K 31/5377; A61K 9/0019; A61K 9/2054; A61K 31/437; A61K 31/44; A61K 31/443; A61K 31/444; A61K 31/519

(57) ABSTRACT

The invention concerns the use of the compound of formula III

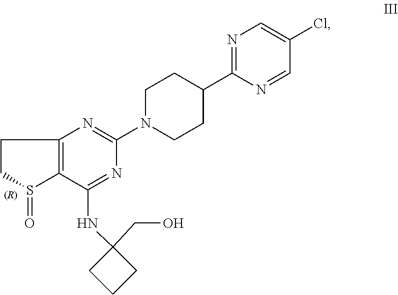

for the manufacture of a pharmaceutical composition comprising 9 mg of the compound of formula III for the treatment of a disease selected from the group consisting of idiopathic pulmonary fibrosis (IPF), progressive pulmonary fibrosis (PPF) and systemic sclerosis (SSc) in a patient suffering from said disease, characterized therein that this pharmaceutical composition comprising 9 mg of the compound of formula III is administered twice daily in combination with or concomitantly to a strong CYP3A-inhibitor (strong P450 3A (CYP3A) inhibitor) to said patient.

### 1. BACKGROUND OF THE INVENTION

### 1.1 Idiopathic Pulmonary Fibrosis (IPF)

Idiopathic pulmonary fibrosis (IPF) is a fibrosing interstitial lung disease characterized by progressive decline in lung function (Raghu G, Remy-Jardin M, Richeldi L, et al. Idiopathic pulmonary fibrosis (an update) and progressive pulmonary fibrosis in adults: an official ATS/ERS/JRS/ALAT clinical practice guideline. Am J Respir Crit Care Med 2022; 205: e18-e47). The pathogenesis of IPF is thought to involve aberrant wound repair in response to alveolar injury, leading to a self-sustaining fibrotic process (Selman M, Pardo A., Cell Signal 2020; 66:109482). Two antifibrotic drugs, nintedanib and pirfenidone, slow decline in lung function in patients with IPF (King T E Jr, Bradford W Z, Castro-Bernardini S, et al., N Engl J Med 2014; 370:2083-2092; Richeldi L, du Bois R M, Raghu G, et al., N Engl J Med 2014; 370:2071-2082) and have become the standard of care. However, these drugs do not halt progression, and their gastro-intestinal side-effects often prevent treatment initiation or result in discontinuation. There remains a need for effective, well-tolerated drugs for IPF that can be used as monotherapy or in combination with other therapies to preserve lung function.

### 1.2 Progressive Pulmonary Fibrosis (PPF)

Progressive pulmonary fibrosis (PPF) is a relatively new term for progressive fibrosing interstitial lung disease (PF-ILD), recommended for use by international guidelines (Rahhu et al, Am J Respir Crit Care Med 2022; 205: e18-e47). Progressive pulmonary fibrosis (PPF) includes any diagnosis of progressive fibrotic interstitial lung disease (ILD) other than idiopathic pulmonary fibrosis (IPF). However, disease progression appears comparable between PPF and IPF, suggesting a similar underlying pathology relating to pulmonary fibrosis. Following positive results in a phase II study in IPF, this phase III study will investigate the efficacy and safety of BI 1015550 in patients with PPF (FIBRONEER-ILD).

### 1.3 Nerandomilast

Nerandomilast (BI 1015550) with the following chemical formula

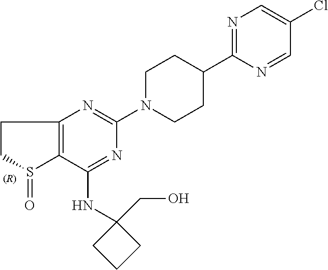

is an orally administered preferential inhibitor of phosphodiesterase 4B (PDE4B). In preclinical models of lung fibrosis, nerandomilast showed antifibrotic and immunomodulatory effects (Herrmann F E, Hesslinger C, Wollin L, Nickolaus P. BI 1015550 is a PDE4B inhibitor and a clinical drug candidate for the oral treatment of idiopathic pulmonary fibrosis. Front Pharmacol 2022; 13:838449. Erratum in: Front Pharmacol 2023; 14:1219760; Reininger D, Fundel-Clemens K, Mayr C H, et al. PDE4B inhibition by nerandomilast: effects on lung fibrosis and transcriptome in fibrotic rats and on biomarkers in human lung epithelial cells. Br J Pharmacol 2024; 181:4766-4781). In a phase 2 trial in 147 patients with IPF, nerandomilast 18 mg twice daily stabilized lung function over 12 weeks, with acceptable safety (Richeldi L, Azuma A, Cottin V, et al. Trial of a preferential phosphodiesterase 4b inhibitor for idiopathic pulmonary fibrosis. N Engl J Med 2022; 386:2178-2187).

WO 2013/026797 (01-2724) discloses nerandomilast as the compound of formula III.

WO2019/081235 (01-3284) discloses a combination of nintedanib and of the PDE4-inhibitor of formula III which shows in an in vitro assay using human lung fibroblasts a synergistic overadditive effect with respect to fibroblast proliferation. However, the in vitro assay used in WO2019/081235 can not address safety and tolerability issues in the human patient and is therefore not suitable to identify appropriate human therapeutic doses or dose regimens.

WO 2023/104958 discloses formulations comprising nerandomilast in the doses of 18 mg and of 9 mg as the only active agent, wherein this formulation is administered twice daily to an IPF-patient. This was supported by showing that 18 mg nerandomilast administered twice daily as the only active agent to an IPF-patient showed efficacy in preventing a loss in FVC/stabilized lung function and showed an acceptable safety profile compared to patients receiving placebo.

WO 2023/104961 discloses the use of 18 mg or 9 mg of nerandomilast administered twice daily in combination with antifibrotic background treatment (consisting of either pirfenidone or nintedanib in authorized dosages) showed efficacy in preventing a loss in FVC/stabilized lung function and showed an acceptable safety profile compared to patients receiving placebo. This was supported by showing that 18 mg nerandomilast administered twice daily in combination to antifibrotic background treatment (consisting either of nintedanib or pirfenidone in authorized dosages) to an IPF-patient showed efficacy in preventing a loss in FVC and showed an acceptable safety profile compared to patients receiving placebo (here: antifibrotic background treatment only).

### 2. DESCRIPTION OF THE INVENTION

In a first aspect the invention concerns an oral pharmaceutical composition comprising
the PDE4B-inhibitor of formula III

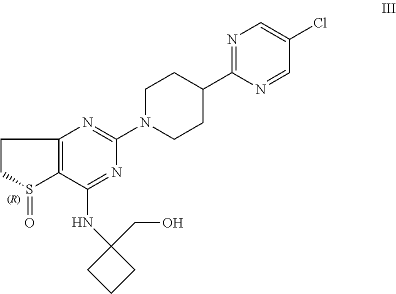

in the dose of 9 mg as the only active agent and
   optionally one or more pharmaceutically acceptable carriers or excipients
for the treatment of a patient suffering from a progressive pulmonary fibrosis (PPF), wherein this oral pharmaceutical composition is to be administered to said patient twice daily.

In a preferred embodiment the progressive pulmonary fibrosis (PPF) is selected from the group consisting of idiopathic non-specific interstitial pneumonia, unclassifiable idiopathic interstitial pneumonia, hypersensitivity pneumonitis, autoimmune interstitial lung disease or other interstitial lungs diseases.

In another preferred embodiment the above-mentioned oral pharmaceutical composition is a film-coated tablet.

In a second aspect the invention concerns the PDE4B-inhibitor of formula III

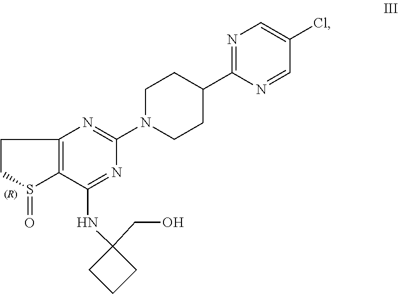

for use in the manufacture of an oral pharmaceutical composition comprising the PDE4B-inhibitor of formula III in the dose of 9 mg as the only active agent and optionally one or more pharmaceutically acceptable carriers or excipients for use in the treatment of a patient suffering from a progressive pulmonary fibrosis (PPF), wherein this oral pharmaceutical composition is to be administered to said patient twice daily.

In a preferred embodiment the progressive pulmonary fibrosis (PPF) is selected from the group consisting of idiopathic non-specific interstitial pneumonia, unclassifiable idiopathic interstitial pneumonia, hypersensitivity pneumonitis, autoimmune interstitial lung disease or other interstitial lungs diseases.

In another preferred embodiment the above-mentioned oral pharmaceutical composition is a film-coated tablet.

In a third embodiment the invention concerns a method of treating a patient suffering from a progressive pulmonary fibrosis (PPF), comprising the step of administering to said patient twice daily an oral pharmaceutical composition comprising the PDE4B-inhibitor of formula III

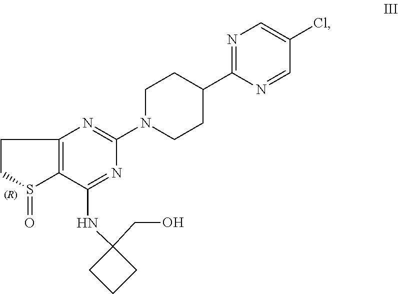

as the only active agent in the dose of 9 mg and optionally one or more pharmaceutically acceptable carriers or excipients.

In a preferred embodiment the progressive pulmonary fibrosis (PPF) is selected from the group consisting of idiopathic non-specific interstitial pneumonia, unclassifiable idiopathic interstitial pneumonia, hypersensitivity pneumonitis, autoimmune interstitial lung disease or other interstitial lungs diseases.

In a further preferred embodiment the above-mentioned oral pharmaceutical composition is a film-coated tablet.

In a fourth aspect the invention concerns an oral pharmaceutical composition comprising.
9 mg of the PDE4B-inhibitor of formula III

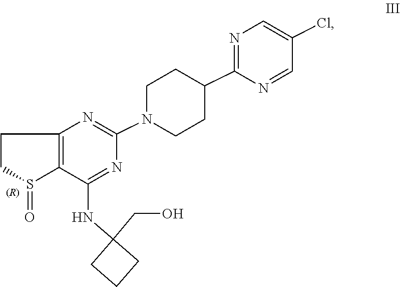

a therapeutically effective dose of nintedanib and
optionally, one or more pharmaceutically acceptable carriers or excipients
for the treatment of a patient suffering from a progressive pulmonary fibrosis (PPF), wherein this oral pharmaceutical composition is to be administered to said patient twice daily.

In a preferred embodiment the progressive pulmonary fibrosis (PPF) is selected from the group consisting of idiopathic non-specific interstitial pneumonia, unclassifiable idiopathic interstitial pneumonia, hypersensitivity pneumonitis, autoimmune interstitial lung disease or other interstitial lungs diseases.

In a further preferred embodiment the above-mentioned oral pharmaceutical composition contains nintedanib in the dose 150 mg.

In a further preferred embodiment the above-mentioned oral pharmaceutical composition contains nintedanib in the dose 100 mg.

In a fifth aspect the invention concerns a method of treating a progressive pulmonary fibrosis (PPF), comprising the step of administering orally to a patient in need thereof 9 mg twice daily of the PDE4B-inhibitor of formula III

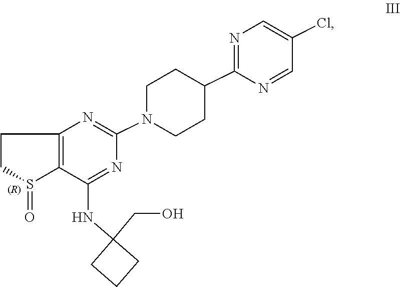

in combination with a therapeutically effective amount of nintedanib.

In a preferred embodiment the progressive pulmonary fibrosis (PPF) is selected from the group consisting of idiopathic non-specific interstitial pneumonia, unclassifiable idiopathic interstitial pneumonia, hypersensitivity pneumonitis, autoimmune interstitial lung disease or other interstitial lungs diseases.

In another preferred embodiment of the above-mentioned method the therapeutically effective amount of nintedanib is 150 mg twice daily.

In another preferred embodiment of the above-mentioned method the therapeutically effective amount of nintedanib is 100 mg twice daily.

In another preferred embodiment of the above-mentioned method the 9 mg of the PDE4B-inhibitor of formula III is administered to the patient in need thereof twice daily in one oral pharmaceutical composition and wherein the therapeutically effective amount of nintedanib is administered to said patient in need thereof in a separate oral pharmaceutical composition.

In another preferred embodiment of the above-mentioned method the 9 mg of the PDE4B-inhibitor of formula III and the therapeutically effective amount of nintedanib are administered together in the same oral pharmaceutical composition twice daily to the patient in need thereof.

In a sixth aspect the invention concerns an oral pharmaceutical composition comprising the PDE4B-inhibitor of formula III

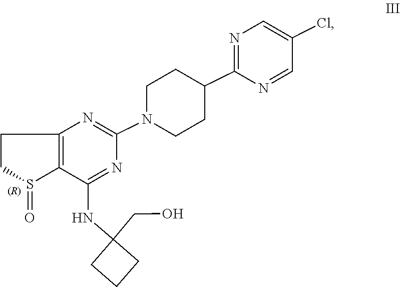

in the dose of 9 mg for use in a method of treating a patient suffering from a progressive pulmonary fibrosis (PPF), wherein said oral pharmaceutical composition comprising 9 mg of the PDE4B-inhibitor of formula III is administered twice daily in combination with a separate oral pharmaceutical composition comprising a therapeutically effective amount of nintedanib.

In a preferred embodiment of the above-mentioned oral pharmaceutical formulation the progressive pulmonary fibrosis (PPF) is selected from the group consisting of idiopathic non-specific interstitial pneumonia, unclassifiable idiopathic interstitial pneumonia, hypersensitivity pneumonitis, autoimmune interstitial lung disease or other interstitial lungs diseases.

In a preferred embodiment of the above-mentioned oral pharmaceutical formulation the therapeutically effective amount of nintedanib is 150 mg twice daily.

In a preferred embodiment of the above-mentioned oral pharmaceutical formulation the therapeutically effective amount of nintedanib is 100 mg twice daily.

In a sixth aspect the invention concerns an oral pharmaceutical composition comprising
the PDE4B-inhibitor of formula III

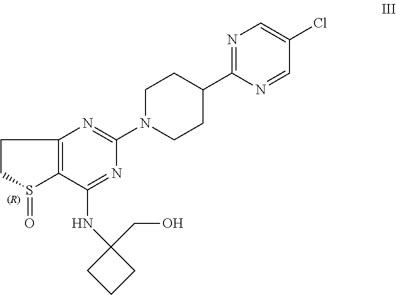

in the dose of 9 mg as the only active agent and
   optionally one or more pharmaceutically acceptable carriers or excipients
for the treatment of a patient suffering from idiopathic pulmonary fibrosis (IPF), wherein this oral pharmaceutical composition is to be administered to said patient twice daily.

In a preferred embodiment the above-mentioned oral pharmaceutical composition is a film-coated tablet.

In a seventh aspect the invention concerns the PDE4B-inhibitor of formula III

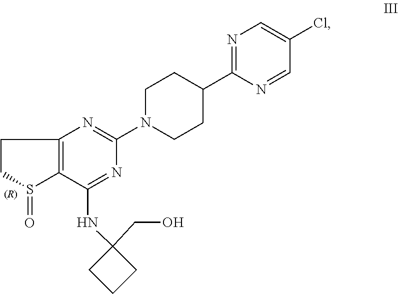

for use in the manufacture of an oral pharmaceutical composition comprising the PDE4B-inhibitor of formula III in the dose of 9 mg as the only active agent and optionally one or more pharmaceutically acceptable carriers or excipients for use in the treatment of a patient suffering from idiopathic pulmonary fibrosis (IPF), wherein this oral pharmaceutical composition is to be administered to said patient twice daily.

In a preferred embodiment the oral pharmaceutical composition is a film-coated tablet.

In an eighth aspect the invention concerns a method of treating a patient suffering from idiopathic pulmonary fibrosis (IPF), comprising the step of administering to said patient twice daily an oral pharmaceutical composition comprising the PDE4B-inhibitor of formula III

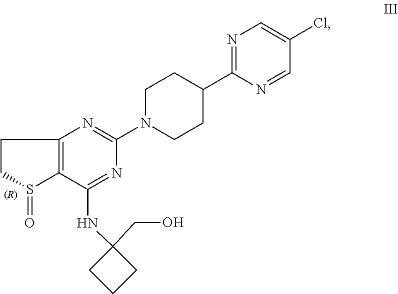

as the only active agent in the dose of 9 mg and optionally one or more pharmaceutically acceptable carriers or excipients.

In a preferred embodiment of the above-mentioned method the oral pharmaceutical composition is a film-coated tablet.

In a ninth aspect the invention concerns an oral pharmaceutical composition comprising.
9 mg of the PDE4B-inhibitor of formula III

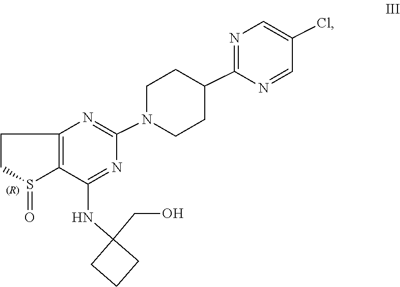

a therapeutically effective dose of nintedanib and
optionally, one or more pharmaceutically acceptable carriers or excipients
for the treatment of a patient suffering from idiopathic pulmonary fibrosis (IPF), wherein this oral pharmaceutical composition is to be administered to said patient twice daily.

In a preferred embodiment the therapeutically effective dose of nintedanib is 150 mg.

In a preferred embodiment the therapeutically effective dose of nintedanib is 100 mg.

In a tenth aspect the invention concerns a method of treating idiopathic pulmonary fibrosis (IPF), comprising the step of administering orally to a patient in need thereof 9 mg twice daily of the PDE4B-inhibitor of formula III

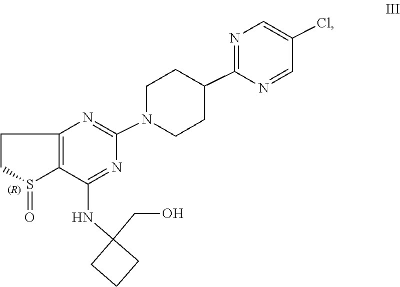

in combination with a therapeutically effective amount of nintedanib.

In a preferred embodiment the therapeutically effective amount of nintedanib is 150 mg twice daily.

In a preferred embodiment the therapeutically effective amount of nintedanib is 100 mg twice daily.

In a preferred embodiment of the above-mentioned method the 9 mg of the PDE4B-inhibitor of formula III is administered to the patient in need thereof twice daily in one oral pharmaceutical composition and wherein the therapeutically effective amount of nintedanib is administered to said patient in need thereof in a separate oral pharmaceutical composition.

In a preferred embodiment of the above-mentioned method the 9 mg of the PDE4B-inhibitor of formula III and the therapeutically effective amount of nintedanib are administered together in the same oral pharmaceutical composition twice daily to the patient in need thereof.

In an eleventh aspect the invention concerns an oral pharmaceutical composition comprising the PDE4B-inhibitor of formula III

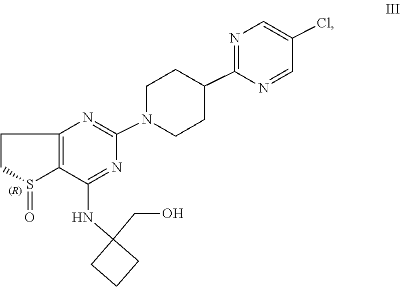

in the dose of 9 mg for use in a method of treating a patient suffering from idiopathic pulmonary fibrosis (IPF), wherein said oral pharmaceutical composition comprising 9 mg of the PDE4B-inhibitor of formula III is administered twice daily in combination with a separate oral pharmaceutical composition comprising a therapeutically effective amount of nintedanib.

In a preferred embodiment of the above-mentioned oral pharmaceutical composition the therapeutically effective amount of nintedanib is 150 mg twice daily.

In a preferred embodiment of the above-mentioned oral pharmaceutical composition the therapeutically effective amount of nintedanib is 100 mg twice daily.

In a twelfth aspect the invention concerns a pharmaceutical composition comprising 9 mg of the compound of formula III

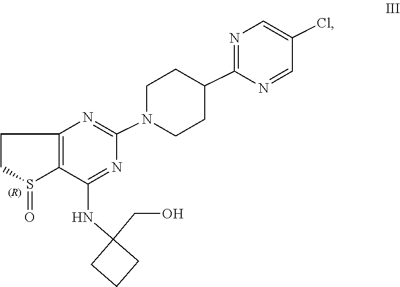

for use in the treatment of a disease selected from the group consisting of idiopathic pulmonary fibrosis (IPF), progressive pulmonary fibrosis (PPF) and systemic sclerosis (SSc), characterized therein that this pharmaceutical composition comprising 9 mg of the compound of formula III is administered to the patient suffering from said disease twice daily and that a strong CYP3A-inhibitor (strong P450 3A (CYP3A) inhibitor) is concomitantly administered to said patient.

In a preferred embodiment of the above-mentioned pharmaceutical composition comprising 9 mg of the compound of formula III for use in the treatment of a disease selected from the group consisting of idiopathic pulmonary fibrosis (IPF), progressive pulmonary fibrosis (PPF) and systemic sclerosis (SSc), this pharmaceutical composition comprising 9 mg of the compound of formula III twice daily and the concomitantly administered strong CYP3A-inhibitor are administered in separate formulations to said patient.

In another preferred embodiment of the above-mentioned pharmaceutical composition comprising 9 mg of the compound of formula III for use in the treatment of a disease selected from the group consisting of idiopathic pulmonary fibrosis (IPF), progressive pulmonary fibrosis (PPF) and systemic sclerosis (SSc), wherein this pharmaceutical composition comprising 9 mg of the compound of formula III is administered to the patient suffering from said disease twice daily concomitantly to said strong CYP3A-inhibitor, wherein this strong CYP3A-inhibitor is selected from the group consisting of itraconazole, ketoconazole, clarithromycin, telithromycin, nefazodone, indinavir, lopinavir, nelfinavir, ritonavir, saquinavir, tipranavir, ceritinib, cobicistat, elvitegravir, idelalisib, paritaprevir, ombitasvir, dasabuvir, posaconazole, voriconazole, adagrasib, boceprevir, danoprevir, ensartinib, ensitrelvir, idelalisib, josamycin, LCL161, lonafarnib, mibefradil, mifepristone, morphothiadin, nirmatrelvir, paritaprevir, posaconazole, relacorilant, ribociclib, telaprevir, troleandomycin, tucatinib, vonoprazan and amoxicillin.

Preferred strong CYP3A-inhibitors are selected from the group consisting of itraconazole, ketoconazole, clarithromycin, telithromycin, nefazodone, indinavir, lopinavir, nelfinavir, ritonavir, saquinavir, tipranavir, ceritinib, cobicistat, elvitegravir, idelalisib, paritaprevir, ombitasvir, dasabuvir, posaconazole and voriconazole, particularly preferred strong CYP3A-inhibitors are selected from the group consisting of traconazole, ketoconazole, clarithromycin, telithromycin, nefazodone, indinavir, lopinavir, nelfinavir, ritonavir, saquinavir and tipranavir.

In another preferred embodiment of the above-mentioned pharmaceutical composition comprising 9 mg of the compound of formula III for use in the treatment of the above-mentioned diseases, the disease is idiopathic pulmonary fibrosis (IPF).

In another preferred embodiment of the above-mentioned pharmaceutical composition comprising 9 mg of the compound of formula III for use in the treatment of the above-mentioned diseases, the disease is progressive pulmonary fibrosis (PPF).

In another preferred embodiment of the above-mentioned pharmaceutical composition comprising 9 mg of the compound of formula III for use in the treatment of the above-mentioned diseases, the disease is systemic sclerosis (SSc).

In a thirteenth aspect, the invention concerns the use of the compound of formula III

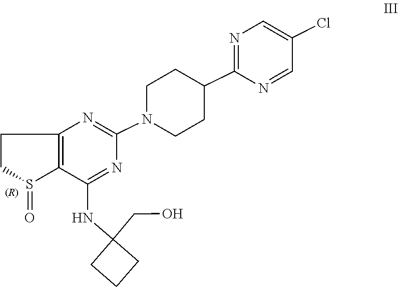

for the manufacture of a pharmaceutical composition comprising 9 mg of the compound of formula III for the treatment of a disease selected from the group consisting of idiopathic pulmonary fibrosis (IPF), progressive pulmonary fibrosis (PPF) and systemic sclerosis (SSc) in a patient suffering from said disease, wherein this pharmaceutical composition comprising 9 mg of the compound of formula III is administered twice daily in combination with or concomitantly to a strong CYP3A-inhibitor (strong P450 3A (CYP3A) inhibitor) to said patient.

In a preferred embodiment of the above-mentioned use of the compound of formula III the pharmaceutical composition comprising 9 mg of the compound of formula III and the concomitantly administered strong CYP3A-inhibitor are administered in separate formulations to said patient.

In another preferred embodiment of the above-mentioned use of the compound of formula III the strong CYP3A-inhibitor is selected from the group consisting of itraconazole, ketoconazole, clarithromycin, telithromycin, nefazodone, indinavir, lopinavir, nelfinavir, ritonavir, saquinavir and tipranavir, ceritinib, cobicistat, elvitegravir, idelalisib, paritaprevir, ombitasvir, dasabuvir, posaconazole, voriconazole, adagrasib, boceprevir, danoprevir, ensartinib, ensitrelvir, idelalisib, josamycin, LCL161, lonafarnib, mibefradil, mifepristone, morphothiadin, nirmatrelvir, paritaprevir, posaconazole, relacorilant, ribociclib, telaprevir, troleandomycin, tucatinib, vonoprazan and amoxicillin.

Preferred strong CYP3A-inhibitors are selected from the group consisting of itraconazole, ketoconazole, clarithromycin, telithromycin, nefazodone, indinavir, lopinavir, nelfinavir, ritonavir, saquinavir, tipranavir, ceritinib, cobicistat, elvitegravir, idelalisib, paritaprevir, ombitasvir, dasabuvir, posaconazole and voriconazole, particularly preferred strong CYP3A-inhibitors are selected from the group consisting of itraconazole, ketoconazole, clarithromycin, telithromycin, nefazodone, indinavir, lopinavir, nelfinavir, ritonavir, saquinavir and tipranavir.

In another preferred embodiment of the above-mentioned use of the compound of formula III the disease is idiopathic pulmonary fibrosis (IPF).

In another preferred embodiment of the above-mentioned use of the compound of formula III the disease is progressive pulmonary fibrosis (PPF)

In another preferred embodiment of the above-mentioned use of the compound of formula III the disease is systemic sclerosis (SSc).

In a fourteenth aspect, the invention concerns a method of treating a patient suffering from a disease selected from the group consisting of idiopathic pulmonary fibrosis (IPF), progressive pulmonary fibrosis (PPF) and systemic sclerosis (SSc), comprising the step of administering to said patient a pharmaceutical composition comprising 9 mg of the compound of formula III

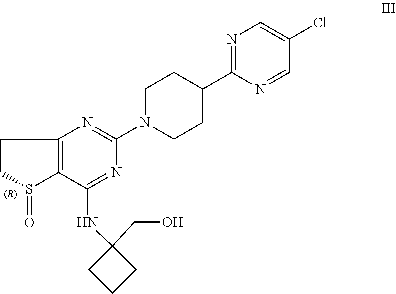

twice daily concomitantly to (or in combination with) a second pharmaceutical composition comprising a strong CYP3A-inhibitor (strong P450 3A (CYP3A) inhibitor).

In a preferred embodiment of that above-mentioned method the strong CYP3A-inhibitor is selected from the group consisting of itraconazole, ketoconazole, clarithromycin, telithromycin, nefazodone, indinavir, lopinavir, nelfinavir, ritonavir, saquinavir and tipranavir, ceritinib, cobicistat, elvitegravir, idelalisib, paritaprevir, ombitasvir, dasabuvir, posaconazole, voriconazole, adagrasib, boceprevir, danoprevir, ensartinib, ensitrelvir, idelalisib, josamycin, LCL161, lonafarnib, mibefradil, mifepristone, morphothiadin, nirmatrelvir, paritaprevir, posaconazole, relacorilant, ribociclib, telaprevir, troleandomycin, tucatinib, vonoprazan and amoxicillin.

Preferred strong CYP3A-inhibitors are selected from the group consisting of itraconazole, ketoconazole, clarithromycin, telithromycin, nefazodone, indinavir, lopinavir, nelfinavir, ritonavir, saquinavir, tipranavir, ceritinib, cobicistat, elvitegravir, idelalisib, paritaprevir, ombitasvir, dasabuvir, posaconazole and voriconazole, particularly preferred strong CYP3A-inhibitors are selected from the group consisting of itraconazole, ketoconazole, clarithromycin, telithromycin, nefazodone, indinavir, lopinavir, nelfinavir, ritonavir, saquinavir and tipranavir.

In a preferred embodiment of that above-mentioned method the patient is suffering from idiopathic pulmonary fibrosis (IPF).

In a preferred embodiment of that above-mentioned method the patient is suffering from progressive pulmonary fibrosis (PPF).

In a preferred embodiment of that above-mentioned method the patient is suffering from systemic sclerosis (SSc).

### 3. EXPERIMENTAL DATA

### 3.1 Fibroneer-IPF Phase III Trial for Idiopathic Pulmonary Fibrosis (IPF)

A phase 3 FIBRONEER-IPF trial was conducted to investigate the efficacy and safety of nerandomilast 9 mg twice daily and 18 mg twice daily, as monotherapy or with background antifibrotic therapy, in patients with IPF.

### 3.1.1 Trial Design and Oversight of the FIBRONEER-IPF Trial for IPF

The FIBRONEER-IPF trial was a randomized, double-blind, placebo-controlled trial conducted at 332 sites in 36 countries (Richeldi L, Azuma A, Cottin V, et al. Design of a phase III, double-blind, randomised, placebo-controlled trial of BI 1015550 in patients with idiopathic pulmonary fibrosis (FIBRONEER-IPF). BMJ Open Respir Res 2023; 10: e001563).

The first patient was screened on 6 Oct. 2022 and the last patient completed the week 52 visit on 16 Aug. 2024. Eligible patients were aged ≥40 years, with IPF, forced vital capacity (FVC) ≥45% of the predicted value and diffusion capacity of the lungs for carbon monoxide (DLco) ≥25% of the predicted value. IPF was diagnosed by the investigator according to guidelines (Raghu et al, Am J Respir Crit Care Med 2022; 205: e18-e47), based on a high-resolution computed tomography (HRCT) scan taken≤12 months prior to screening. A usual interstitial pneumonia (UIP) or probable UIP pattern on HRCT was confirmed by central review. Patients with an indeterminate pattern on HRCT or a pattern suggesting an alternative diagnosis could participate if IPF was confirmed locally based on historical surgical lung biopsy or cryobiopsy.

Patients taking nintedanib or pirfenidone must have taken a stable dose for ≥12 weeks. Patients not treated with antifibrotic therapy for ≥8 weeks were eligible to participate; these patients could initiate nintedanib or pirfenidone in the event of IPF progression and/or acute exacerbation after 12 weeks of the trial. Prednisone >15 mg/day or equivalent for respiratory reasons, not permitted at enrollment, could be prescribed during the trial for suspected acute exacerbation.

Patients were randomized 1:1:1 to receive nerandomilast 9 mg twice daily, nerandomilast 18 mg twice daily, or placebo twice daily using interactive response technology. Randomization was stratified by use of background antifibrotic therapy (nintedanib/pirfenidone vs none). Visits occurred at baseline, weeks 2, 6, 12, 18, 26, 36, 44 and 52, and every 12 weeks thereafter. A follow-up visit was performed 7 days after end of treatment. Interruption of nerandomilast was permitted to manage adverse events. Dose reductions and interruptions of background nintedanib or pirfenidone were recommended to manage adverse events, as per product labels. Patients who prematurely discontinued nerandomilast were asked to attend visits as planned. Here we present data collected until first database lock, which took place after the last patient had completed the week 52 visit. Patients continued blinded treatment until final database lock. Patients who completed the trial on treatment were eligible to receive open-label nerandomilast in an extension study.

The primary end point was absolute change from baseline in FVC (ml) at week 52. Spirometry was conducted using SpiroSphere (Clario) spirometers in accordance with the recommendations of the American Thoracic Society and European Respiratory Society (Graham et al, Am J Respir Crit Care Med 2019; 200: e70-e88). The key secondary end point was time to first acute exacerbation, hospitalization for respiratory cause, or death, over the duration of the trial. These endpoints were not adjudicated. Acute exacerbations were defined as per published guidelines (Collard et al, Am J Respir Crit Care Med 2016; 194:265-275). The secondary endpoints were absolute change in FVC percentage of the predicted value at week 52; absolute change in DLco percentage of the predicted value at week 52; absolute changes in the Living with Pulmonary Fibrosis questionnaire, dyspnea score, cough score and fatigue scores (for which higher scores on a scale of 0 to 100 indicate worse symptoms) at week 52; time to acute exacerbation or death, time to hospitalization for respiratory cause or death, time to absolute decline in FVC percentage of the predicted value >10% or death, time to absolute decline in DLco percentage of the predicted value >15% or death, and time to death over the duration of the trial. DLco was measured using the site's equipment in accordance with recommendations from the American Thoracic Society and European Respiratory Society (Macintyre et al, Eur Respir J 2005; 26:720-735). Time to initiation of supplemental oxygen was a pre-specified further endpoint. Plasma concentrations of nerandomilast at steady state were determined by high performance liquid chromatography with tandem mass spectrometry.

Safety was assessed through recording of adverse events and clinical monitoring. Adverse events were coded according to the Medical Dictionary for Regulatory Activities (MedDRA) v27.0. Adverse events of interest for PDE4 inhibitors, such as vasculitis, depression, and suicidality were assessed.

### 3.1.2 Efficacy Results of the FIBRONEER-IPF Trial for IPF

A total of 1177 randomized patients received at least one dose of trial drug. Baseline characteristics were similar across treatment groups (Table 1).

TABLE 1
Characteristics of patients at baseline*

| | | Nerandomilast | Nerandomilast |
|---|---|---|---|
| | | 9 mg twice | 18 mg twice |
| | Placebo | daily | daily |
| | (N = 393) | (N = 392) | (N = 392) |
| Male sex, no. | 337 | 317 | 323 |
| (%) | (85.8) | (80.9) | (82.4) |
| Age, years | 69.9 ± 7.5 | 70.5 ± 7.8 | 70.3 ± 7.8 |
| Weight, kg | 77.5± 14.5 | 76.1 ± 15.8 | 76.3 ± 14.0 |
| Smoking status, | | | |
| no. (%) | | | |
| Never smoked | 113 | 119 | 109 |
| | (28.8) | (30.4) | (27.8) |
| Former smoker | 275 | 263 | 272 |
| | (70.0) | (67.1) | (69.4) |
| Current smoker | 5 | 10 | 11 |
| | (1.3) | (2.6) | (2.8) |
| Time since | 3.5 ± 2.7 | 3.5 ± 2.6 | 3.6 ± 2.8 |
| diagnosis of | | | |
| IPF, years | | | |
| FVC | | | |
| Mean value - | 2864 ± 805 | 2837 ± 781 | 2827 ± 758 |
| ml | | | |
| Percentage of | 77.3 ± 18.3 | 79.0 ± 16.7 | 78.4 ± 16.8 |
| predicted value | | | |
| DLco, | 49.4 ± 15.8 | 51.7 ± 15.5 | 51.5 ± 17.5 |
| percentage of | | | |
| predicted value | | | |
| Background | | | |
| antifibrotic | | | |
| treatment, | | | |
| no. (%) | | | |
| Nintedanib | 173 | 184 | 178 |
| | (44.0) | (46.9) | (45.4) |
| Pirfenidone | 133 | 120 | 127 |
| | (33.8) | (30.6) | (32.4) |
| None | 87 | 88 | 87 |
| | (22.1) | (22.4) | (22.2) |
| Supplemental | 90 | 68 | 90 |
| oxygen, | (22.9) | (17.3) | (23.0) |
| no. (%) | | | |

*Plus-minus values are means ± SD
^{†}The values for DLco were corrected for hemoglobin level. Data were available for 391 patients in the placebo group, 391 patients in the nerandomilast 9 mg twice daily group and 389 patients in the nerandomilast 18 mg twice daily group

Mean (±SD) age was 70.2±7.7 years, FVC was 78.2±17.3% of the predicted value and DLco was 50.9±16.3% of the predicted value. The trial population reflected the characteristics of the general population of patients with IPF. A total of 915 patients (77.7%) took background antifibrotic therapy (535 nintedanib, 380 pirfenidone).

Similar proportions of patients in each treatment group (81.1%-82.7%) completed 52 weeks of treatment (FIG. 1). and completed treatment until first database lock. Mean (±SD) exposure to trial medication until first database lock was approximately 13 months in each group.

Adjusted mean changes in FVC (ml) at week 52 were −183.5 (95% CI, −210.9 to −156.1) in the placebo group, −138.6 (95% CI, −165.6 to −111.6) in the nerandomilast 9 mg twice daily group and −114.7 (95% CI, −141.8 to −87.5) in the nerandomilast 18 mg twice daily group (FIG. 1). The difference vs placebo was 44.9 (95% confidence interval [CI], 6.4 to 83.3; P=0.02) for nerandomilast 9 mg twice daily and 68.8 (95% CI, 30.3 to 107.4; P<0.001) for nerandomilast 18 mg twice daily. The FVC curves separated shortly after randomization and continued to diverge up to week 52.

Among patients not taking background antifibrotic therapy, adjusted mean changes in FVC (ml) at week 52 were −148.7 in the placebo group, −70.4 in the nerandomilast 9 mg twice daily group and −79.2 in the nerandomilast 18 mg twice daily group (FIG. 2) Among patients taking nintedanib, adjusted mean changes in FVC (ml) at week 52 were −191.6 in the placebo group, −130.7 in the nerandomilast 9 mg twice daily group and −118.5 in the nerandomilast 18 mg twice daily group (FIG. 3). Among patients taking pirfenidone, adjusted mean changes in FVC (ml) at week 52 were −197.0 in the placebo group, −201.8 in the nerandomilast 9 mg twice daily group and −133.7 in the nerandomilast 18 mg twice daily group (FIG. 4).

Pre-specified subgroup analyses did not indicate heterogeneity in the effect of nerandomilast versus placebo across subgroups, except that no effect of nerandomilast 9 mg twice daily was observed in patients taking pirfenidone.

The key secondary end point was experienced by 80 patients (20.4%) in the placebo group, 79 patients (20.2%) in the nerandomilast 9 mg twice daily group and 85 patients (21.7%) in the nerandomilast 18 mg twice daily group. The hazard ratio was 1.03 (95% CI, 0.75 to 1.41; P=0.86) for nerandomilast 9 mg twice daily vs placebo and 1.17 (95% CI, 0.86 to 1.59; P=0.31) for nerandomilast 18 mg twice daily vs placebo. The other time-to-event end points are shown in Table 2 (secondary endpoints).

TABLE 2
Time-to-event end points up to first database lock

| | | Nerandomilast | Nerandomilast |
|---|---|---|---|
| | Placebo | 9 mg twice | 18 mg twice |
| | (N = | daily (N = | daily (N = |
| | 393) | 392) | 392) |
| Key secondary | | | |
| end point | | | |
| First acute | 80 | 79 | 85 |
| exacerbation, | (20.4) | (20.2) | (21.7) |
| hospitalization | | | |
| for respiratory | | | |
| cause, or death, | | | |
| no. with | | | |
| event (%) | | | |
| Difference vs | | 1.03 | 1.17 |
| placebo, HR | | (0.75 | (0.86 |
| (95% CI) | | to | to |
| | | 1.41) | 1.59) |
| P Value vs | | 0.86 | 0.31 |
| placebo | | | |
| Secondary | | | |
| time-to-event | | | |
| end points | | | |
| Acute | 49 | 51 | 50 |
| exacerbation | (12.5) | (13.0) | (12.8) |
| or death, no. | | | |
| with | | | |
| event (%) | | | |
| Difference vs | | 1.12 | 1.11 |
| placebo, HR | | (0.76 | (0.75 |
| (95% CI) | | to | to |
| | | 1.67) | 1.65) |
| Hospitalization | 73 | 68 | 75 |
| for respiratory | (18.6) | (17.3) | (19.1) |
| cause or death, | | | |
| no. with event | | | |
| (%) | | | |
| Difference vs | | 0.98 | 1.13 |
| placebo, HR | | (0.70 | (0.82 |
| (95% CI) | | to | to |
| | | 1.36) | 1.56) |
| Death, no. | 28 | 26 | 21 |
| with event | (7.1) | (6.6) | (5.4) |
| (%) | | | |
| Difference vs | | 1.03 | 0.81 |
| placebo, HR | | (0.60 | (0.46 |
| (95% CI) | | to | to |
| | | 1.76) | 1.43) |
| Absolute | 111 | 107 | 94 |
| decline in FVC | (28.2) | (27.3) | (24.0) |
| >10% of the | | | |
| predicted value | | | |
| from baseline | | | |
| or death, | | | |
| no. with event | | | |
| (%) | | | |
| Difference vs | | 0.96 | 0.84 |
| placebo, HR | | (0.74, | (0.64, |
| (95% CI) | | 1.25) | 1.10) |
| Absolute | 66 | 59 | 59 |
| decline DLco | (16.8) | (15.1) | (15.1) |
| >15% of the | | | |
| predicted value | | | |
| from baseline | | | |
| or death, | | | |
| no. with event | | | |
| (%) | | | |
| Difference vs | | 0.98 | 0.88 |
| placebo, HR | | (0.69, | (0.62, |
| (95% CI) | | 1.41) | 1.26) |

Among patients not using supplemental oxygen at enrollment, 58 (19.5%), 38 (12.1%) and 48 (16.0%) patients in the placebo, nerandomilast 9 mg twice daily and nerandomilast 18 mg twice daily groups, respectively, initiated oxygen during the trial.

### 3.1.3 Adverse Events-Results of the FIBRONEER-IPF Trial for IPF

The percentages of patients with any adverse events and any serious adverse events over 52 weeks were similar across treatment groups (Table 3). The most frequent adverse event was diarrhea, reported in 16.0% of the placebo group, 31.1% of the nerandomilast 9 mg twice daily group and 41.3% of the nerandomilast 18 mg twice daily group. Rates of diarrhea were higher in patients taking background antifibrotic therapy than not (Table 3). Among the patients who had diarrhea adverse events, the worst event was severe in 1.6%, 4, 1%, 5.6% of patients in the placebo, nerandomilast 9 mg twice daily and nerandomilast 18 mg twice daily groups, respectively. Adverse events in patients taking background nintedanib and background pirfenidone are shown in Table 3.

TABLE 3
Adverse events over 52 weeks*

| All patients | | | No background antifibrotic therapy | | |
|---|---|---|---|---|---|
| | Nerandomilast | Nerandomilast | | Nerandomilast | Nerandomilast |
| Placebo | 9 mg twice | 18 mg twice | Placebo | 9 mg twice | 18 mg twice |
| (N = 393) | daily (N = 392) | daily (N = 392) | (N = 87) | daily (N = 88) | daily (N = 87) |

No. of patients (%)

| | | | | | | | | | | | | |
|---|---|---|---|---|---|---|---|---|---|---|---|---|
| Adverse event | 371 | (94.4) | 364 | (92.9) | 372 | (94.9) | 83 | (95.4) | 77 | (87.5) | 79 | (90.8) |
| Most frequent | | | | | | | | | | | | |
| adverse events^{†} | | | | | | | | | | | | |
| Diarrhea | 63 | (16.0) | 122 | (31.1) | 162 | (41.3) | 7 | (8.0) | 15 | (17.0) | 23 | (26.4) |
| Cough | 65 | (16.5) | 65 | (16.6) | 55 | (14.0) | 4 | (4.6) | 12 | (13.6) | 9 | (10.3) |
| COVID-19 | 45 | (11.5) | 59 | (15.1) | 51 | (13.0) | 5 | (5.7) | 8 | (9.1) | 14 | (16.1) |
| Upper | 40 | (10.2) | 39 | (9.9) | 49 | (12.5) | 8 | (9.2) | 10 | (11.4) | 13 | (14.9) |
| respiratory | | | | | | | | | | | | |
| tract infection | | | | | | | | | | | | |
| Dyspnea | 50 | (12.7) | 39 | (9.9) | 35 | (8.9) | 12 | (13.8) | 2 | (2.3) | 6 | (6.9) |
| Nasopharyngitis | 44 | (11.2) | 40 | (10.2) | 35 | (8.9) | 12 | (13.8) | 9 | (10.2) | 8 | (9.2) |
| Condition | 40 | (10.2) | 32 | (8.2) | 38 | (9.7) | 10 | (11.5) | 3 | (3.4) | 7 | (8.0) |
| aggravated | | | | | | | | | | | | |
| Weight | 30 | (7.6) | 35 | (8.9) | 40 | (10.2) | 5 | (5.7) | 1 | (1.1) | 6 | (6.9) |
| decreased | | | | | | | | | | | | |
| Adverse event | 42 | (10.7) | 46 | (11.7) | 55 | (14.0) | 7 | (8.0) | 7 | (8.0) | 6 | (6.9) |
| leading to | | | | | | | | | | | | |
| treatment | | | | | | | | | | | | |
| discontinuation | | | | | | | | | | | | |
| Diarrhea leading | 2 | (0.5) | 7 | (1.8) | 24 | (6.1) | 0 | (0) | 0 | (0) | 1 | (1.1) |
| to treatment | | | | | | | | | | | | |
| discontinuation | | | | | | | | | | | | |
| Adverse event | 56 | (14.2) | 72 | (18.4) | 76 | (19.4) | 13 | (14.9) | 12 | (13.6) | 11 | (12.6) |
| leading to | | | | | | | | | | | | |
| treatment | | | | | | | | | | | | |
| interruption | | | | | | | | | | | | |
| Serious adverse | 131 | (33.3) | 121 | (30.9) | 117 | (29.9) | 28 | (32.2) | 22 | (25.0) | 20 | (23.0) |
| event^{‡} | | | | | | | | | | | | |
| Fatal adverse | 18 | (4.6) | 16 | (4.1) | 6 | (1.5) | 7 | (8.0) | 3 | (3.4) | 0 | (0) |
| event | | | | | | | | | | | | |

| | Background nintedanib therapy | | | | | | Background pirfenidone therapy | | | | | |
|---|---|---|---|---|---|---|---|---|---|---|---|---|
| | | | Nerandomilast | | Nerandomilast | | | | Nerandomilast | | Nerandomilast | |
| | | | 9 mg twice | | 18 mg twice | | | | 9 mg twice | | 18 mg twice | |
| | Placebo | | daily | | daily | | Placebo | | daily | | daily | |
| | (N = 173) | | (N = 184) | | (N = 178) | | (N = 133) | | (N = 120) | | (N = 127) | |
| | No. of patients (%) | | | | | | | | | | | |
| Adverse event | 163 | (94.2) | 175 | (95.1) | 174 | (97.8) | 125 | (94.0) | 112 | (93.3) | 119 | (93.7) |
| Most frequent | | | | | | | | | | | | |
| adverse events^{†} | | | | | | | | | | | | |
| Diarrhea | 46 | (26.6) | 91 | (49.5) | 110 | (61.8) | 10 | (7.5) | 16 | (13.3) | 29 | (22.8) |
| Cough | 31 | (17.9) | 28 | (15.2) | 21 | (11.8) | 30 | (22.6) | 25 | (20.8) | 25 | (19.7) |
| COVID-19 | 21 | (12.1) | 30 | (16.3) | 17 | (9.6) | 19 | (14.3) | 21 | (17.5) | 20 | (15.7) |
| Upper | 14 | (8.1) | 17 | (9.2) | 24 | (13.5) | 18 | (13.5) | 12 | (10.0) | 12 | (9.4) |
| respiratory | | | | | | | | | | | | |
| tract infection | | | | | | | | | | | | |
| Dyspnea | 23 | (13.3) | 21 | (11.4) | 14 | (7.9) | 15 | (11.3) | 16 | (13.3) | 15 | (11.8) |
| Nasopharyngitis | 19 | (11.0) | 19 | (10.3) | 17 | (9.6) | 13 | (9.8) | 12 | (10.0) | 10 | (7.9) |
| Condition | 17 | (9.8) | 16 | (8.7) | 18 | (10.1) | 13 | (9.8) | 13 | (10.8) | 13 | (10.2) |
| aggravated | | | | | | | | | | | | |
| Weight | 19 | (11.0) | 24 | (13.0) | 28 | (15.7) | 6 | (4.5) | 10 | (8.3) | 6 | (4.7) |
| decreased | | | | | | | | | | | | |
| Adverse event | 22 | (12.7) | 31 | (16.8) | 38 | (21.3) | 13 | (9.8) | 8 | (6.7) | 11 | (8.7) |
| leading to | | | | | | | | | | | | |
| treatment | | | | | | | | | | | | |
| discontinuation | | | | | | | | | | | | |
| Diarrhea leading | 2 | (1.2) | 4 | (2.2) | 23 | (12.9) | 0 | (0) | 3 | (2.5) | 0 | (0) |
| to treatment | | | | | | | | | | | | |
| discontinuation | | | | | | | | | | | | |
| Adverse event | 21 | (12.1) | 45 | (24.5) | 48 | (27.0) | 22 | (16.5) | 15 | (12.5) | 17 | (13.4) |
| leading to | | | | | | | | | | | | |
| treatment | | | | | | | | | | | | |
| interruption | | | | | | | | | | | | |
| Serious adverse | 50 | (28.9) | 62 | (33.7) | 56 | (31.5) | 53 | (39.8) | 37 | (30.8) | 41 | (32.3) |
| event^{‡} | | | | | | | | | | | | |
| Fatal adverse | 5 | (2.9) | 9 | (4.9) | 4 | (2.2) | 6 | (4.5) | 4 | (3.3) | 2 | (1.6) |
| event | | | | | | | | | | | | |

*Listed are adverse events that were reported over the 52-week trial period or until 7 days after the last dose in patients who discontinued treatment before week 52.
^{†}These events were coded based on preferred terms in the Medical Dictionary for Regulatory Activities. Adverse events that were reported in more than 10% of the patients in any of the treatment groups in the overall population are shown.
^{‡}A serious adverse event was defined as an event that resulted in death, in hospitalization or prolongation of hospitalization, or in persistent or clinically significant disability or incapacity; or was life-threatening, a congenital anomaly or birth defect, or deemed to be serious for any other reason.

Over 52 weeks, adverse events led to interruption of trial drug in 14.2% of the placebo group, 18.4% of the nerandomilast 9 mg twice daily group and 19.4% of the nerandomilast 18 mg twice daily group. Adverse events led to permanent discontinuation of trial drug in 10.7% of the placebo group, 11.7% of the nerandomilast 9 mg twice daily group and 14.0% of the nerandomilast 18 mg twice daily group. The adverse event that most frequently led to treatment discontinuation was diarrhea, which led to discontinuation in 0.5%, 1.8% and 6.1% of these groups, respectively. Adverse events led to treatment discontinuation more frequently among patients taking background antifibrotic therapy across treatment groups (Table 3).

The adverse events reported up to first database lock were consistent with those reported over 52 weeks. There were no imbalances between the nerandomilast and the placebo groups in adverse events of interest such as vasculitis, depression, suicidality, or drug-induced liver injury.

### 3.1.4 Plasma Concentrations of Nerandomilast in the FIBRONEER-IPF Trial

Over 26 weeks, mean trough plasma concentrations of nerandomilast at pharmacokinetic steady state were approximately 50% lower in patients taking pirfenidone than in patients taking nintedanib or not taking background antifibrotic therapy as it can be seen from FIG. 5. This effect was due to an interaction between nerandomilast and pirfenidone which reduced plasma concentrations of nerandomilast by about 50% in patients taking pirfenidone and meant that only the 18 mg twice daily dose of nerandomilast was effective in these patients. FIG. 5 shows that in patients that 18 mg twice daily (bid) nerandomilast concomitantly with pirfenidone, showed due to that interaction a 50% reduction in the nerandomilast plasma concentration in comparison to those patients that received 18 mg twice daily nerandomilast either alone or concomitantly with nintedanib, however the remaining nerandomilast plasma concentration (around 100 nmol/L) was still similar to the plasma concentration in patients that had received 9 mg twice daily nerandomilast either alone or concomitantly with nintedanib (which was proven to be an efficacious dose; see FIG. 3). Consequently, the therapeutic effect of 9 mg twice daily nerandomilast either administered without antifibrotic background therapy or concomitantly with nerandomilast seems to be similar to the therapeutic effect of 18 mg twice daily nerandomilast administered concomitantly with pirfenidone. In contrast to that FIG. 5 shows clearly that the low remaining plasma concentration of nerandomilast in patients that received pirfenidone as concomitant antifibrotic background therapy after the 50% reduction due to the pirfenidone/nerandomilast interaction is obviously sub-therapeutic and therefore no longer efficacious (as shown in FIG. 4).

### 3.2 Fibroneer-ILD Phase III Trial (for Progressive Pulmonary Fibrosis (PPF))

Further, a phase 3 FIBRONEER-ILD trial to investigate the efficacy and safety of nerandomilast 9 mg twice daily and 18 mg twice daily, as monotherapy or with background antifibrotic therapy in patients with progressive pulmonary fibrosis (PPF) (background antifibrotic therapy in PPF: nintedanib).

### 3.2.1 Trial Design and Oversight of the FIBRONEER-ILD Trial for Progressive Pulmonary Fibrosis (PPF))

The Fibroneer-ILD trial was a phase III, randomized, double-blind, placebo-controlled, multicenter study in 44 countries worldwide (Maher et al BMJ Open Respir Res 2023; 10: e0011563).

In this phase III, double-blind, placebo-controlled trial, patients were randomised 1:1:1 to receive nerandomilast (9 mg or 18 mg) or placebo twice daily over at least 52 weeks, stratified by background nintedanib use. Patients had to be diagnosed with pulmonary fibrosis other than IPF that is progressive, based on predefined criteria. Patients had to have forced vital capacity (FVC) ≥45% predicted and haemoglobin-corrected diffusing capacity of the lung for carbon monoxide ≥25% predicted. Patients had to be receiving nintedanib for at least 12 weeks, or not receiving nintedanib for at least 8 weeks, prior to screening. Patients on stable treatment with permitted immunosuppressives (eg, methotrexate, azathioprine) were allowed to continue their treatment throughout the trial. Patients with clinically significant airway obstruction or other pulmonary abnormalities, and those using immunosuppressives that may confound FVC results (cyclophosphamide, tocilizumab, mycophenolate, rituximab) or high-dose steroids were excluded. The primary endpoint was defined as the absolute change from baseline in FVC (mL) at week 52. The key secondary endpoint was defined to be the time to the first occurrence of any acute ILD exacerbation, hospitalisation for respiratory cause or death, over the duration of the trial.

### 3.2.2 Efficacy Results of the FIBRONEER-ILD Trial for Progressive Pulmonary Fibrosis (PPF))

A total of 1176 randomized patients received at least one dose of trial drug. Baseline characteristics were similar across treatment groups (Table 4a-c).

TABLE 4a
Fibroneer-ILD: Baseline characteristics:

| | | Nera 9 | Nera 18 |
|---|---|---|---|
| | Placebo | mg bid | mg bid |
| Variables | (n = 392) | (n = 393) | (n = 391) |
| Male, | 231 | 203 | 220 |
| n (%) | (58.9) | (51.7) | (56.3) |
| Race, n (%) | | | |
| White | 226 | 224 | 235 |
| | (57.7) | (57.0) | (60.1) |
| Asian | 149 | 155 | 143 |
| | (38.0) | (39.4) | (36.6) |
| Black/ | 7 | 3 | 6 |
| African- | (1.8) | (0.8) | (1.5) |
| American | | 6 | 2 |
| Other | 5 | (1.5) | (0.5) |
| | (1.3) | | |
| Age, years, | 66.6 | 66.5 | 66.0 |
| mean (SD) | (10.3) | (9.8) | (9.8) |
| Age category, | | | |
| n (%) | | | |
| <65 | 149 | 142 | 152 |
| | (38.0) | (36.1) | (38.9) |
| ≥65 | 243 | 251 | 239 |
| | (62.0) | (63.9) | (61.1) |
| Weight, kg, | 73.4 | 72.1 | 73.2 |
| mean (SD) | (17.9) | (17.5) | (17.1) |
| Smoking | | | |
| status, n (%) | | | |
| Never | 186 | 200 | 191 |
| | (47.4) | (50.9) | (48.8) |
| Former | 200 | 186 | 189 |
| | (51.0) | (47.3) | (48.3) |
| Current | 6 | 7 | 11 |
| | (1.5) | (1.8) | (2.8) |

TABLE 4b
Fibroneer-ILD: Baseline characteristics
related to PF-ILD

| | | Nera 9 | Nera 18 |
|---|---|---|---|
| | Placebo | mg bid | mg bid |
| Variables | (n = 392) | (n = 393) | (n = 391) |
| Time | | | |
| since | | | |
| ILD | | | |
| diagnosis, | | | |
| years | | | |
| Mean | 3.9 | 4.1 | 4.6 |
| (SD) | (3.6) | (4.3) | (4.8) |
| Median | 2.7 | 2.6 | 3.2 |
| (IQR) | (1.5-5.7) | (1.1-5.8) | (1.5-6.1) |
| Supple | 110 | 97 | 117 |
| mental | (28.1) | (24.7) | (29.9) |
| oxygen | | | |
| use, n | | | |
| (%) | | | |
| FVC, | 2354 | 2326 | 2381 |
| mL, | (766) | (768) | (723) |
| (mean, | | | |
| SD) | | | |
| FVC, % | 69.7 | 70.3 | 70.4 |
| predicted, | (16.2) | (15.7) | (15.5) |
| mean | | | |
| (SD) | | | |
| DLco, % | 49.7 | 48.7 | 49.4 |
| predicted, | (16.5) | (16.8) | (17.5) |
| mean | | | |
| (SD)* | | | |
| Meet PPF | 344 | 341 | 340 |
| Criteria, n | (87.8) | (86.8) | (87.0) |
| (%) | | | |

TABLE 4c
Fibroneer-ILD: Baseline characteristics
related to PF-ILD-continued

| | | Nera 9 | Nera 18 |
|---|---|---|---|
| | Placebo | mg bid | mg bid |
| Variables | (n = 392) | (n = 393) | (n = 391) |
| Underlying | | | |
| Clinical | | | |
| ILD | | | |
| diagnosis in | | | |
| groups, n | | | |
| (%) | | | |
| Idiopathic | 73 | 73 | 82 |
| nonspecific | (18.6) | (18.6) | (21.0) |
| interstitial | | | |
| pneumonia | | | |
| Unclassifiable | 82 | 76 | 73 |
| idiopathic | (20.9) | (19.3) | (18.7) |
| interstitial | | | |
| pneumonia | | | |
| Hypersensitivity | 77 | 83 | 73 |
| pneumonitis | (19.6) | (21.1) | (18.7) |
| Autoimmune | 100 | 112 | 113 |
| ILD | (25.5) | (28.5) | (28.9) |
| Other ILD | 60 | 49 | 50 |
| | (15.3) | (12.5) | (12.8) |
| HRCT | 275 | 290 | 275 |
| pattern = | (70.2) | (73.8) | (70.3) |
| UIP or | | | |
| UIP-like, n | | | |
| (%) | | | |

Adjusted mean changes in FVC (ml) at week 52 were −165.8 (95% CI, −109.5 to −141.0) in the placebo group, −84.6 (95% CI, −109.6 to −59.7) in the nerandomilast 9 mg twice daily group and −98.6 (95% CI, −123.7 to −73.4) in the nerandomilast 18 mg twice daily group (FIG. 6 and Table 5). The difference vs placebo was 81.1 (95% confidence interval [CI], 46.0 to 116.3; P<0.0001) for nerandomilast 9 mg twice daily (relative reduction of 49%) and 67.2 (95% CI, 31.9 to 102.5; P<0.0002) for nerandomilast 18 mg twice daily (relative reduction of 41%). The FVC curves separated shortly after randomization and continued to diverge up to week 52 (see FIG. 7).

Hereby, the primary endpoint (change from baseline in Forced Vital Capacity (FVC) in mL at week 52 had been met statistically significantly for both doses, 18 mg nerandomilast twice daily and 9 mg twice daily.

TABLE 5
Primary endpoint: change from baseline in Forced
Vital Capacity (FVC) (mL) at week 52

| | | Nera 9 | Nera 18 |
|---|---|---|---|
| | Placebo | mg bid | mg bid |
| | (n = 392) | (n = 393) | (n = 391) |
| N | 391 | 390 | 390 |
| analyzed | | | |
| Baseline | 2353.6 | 2325.5 | 2381.4 |
| FVC | (766.2) | (767.6) | (722.8) |
| [mL] | | | |
| mean | | | |
| (SD) | | | |
| Adjusted | −165.8 | −84.6 | −98.6 |
| mean | (−190.5, | (−109.6, | (−123.7, |
| (95% CI) | −141.0) | −59.7) | −73.4) |
| Adjusted | — | 81.1 | 67.2 |
| mean diff. | | (46.0, | (31.9, |
| vs placebo | | 116.3) | 102.5) |
| (95% | | | |
| CI) | | | |
| p-value | — | <0.0001* | 0.0002* |

Among patients not taking background antifibrotic therapy, adjusted mean changes in FVC (ml) at week 52 were −154.1 (−187.1, −121.2) in the placebo group, −82.3 (−115.9, −48.8) in the nerandomilast 9 mg twice daily group and −95.2 (−128.6, −61.9) in the nerandomilast 18 mg twice daily group (see Table 6).

Among patients taking background antifibrotic therapy by nintedanib, adjusted mean changes in FVC (ml) at week 52 were −180.9 (−218.6, −143.2) in the placebo group, −87.8 (−125.4, −50.2) in the nerandomilast 9 mg twice daily group and −102.9 (−141.2, −64.5) in the nerandomilast 18 mg twice daily group (see Table 6).

This shows that there are relatively consistent efficacies of both doses of nerandomilast (18 mg twice daily and 9 mg twice daily) observed in patients without antifibrotic background therapy and with antifibrotic background therapy with nintedanib in progressive pulmonary fibrosis.

TABLE 6
Change from baseline in FVC (mL) at week 52 by treatment and in the presence
or absence of background antifibrotic therapy (with nintedanib)

| | Background AF therapy | | | No background AF therapy | | |
|---|---|---|---|---|---|---|
| | Placebo | Nera 9 mg | Nera 18 mg | Placebo | Nera 9 mg | Nera 18 mg |
| | (n = 170) | bid (n = 173) | bid (n = 171) | (n = 222) | bid (n = 220) | bid (n = 220) |
| N analyzed | 169 | 173 | 170 | 222 | 217 | 220 |
| Baseline | 2471 | 2430 | 2417 | 2264 | 2244 | 2354 |
| FVC, mean | (786.3) | (768.7) | (733.4) | (739.8) | (758.6) | (715.0) |
| (SD) | | | | | | |
| Adjusted | −180.9 | −87.8 | −102.9 | −154.1 | −82.3 | −95.2 |
| mean (95% | (−218.6, −143.2) | (−125.4, −50.2) | (−141.2, −64.5) | (−187.1, −121.2) | (−115.9, −48.8) | (−128.6, −61.9) |
| CI) change | | | | | | |
| from | | | | | | |
| baseline in | | | | | | |
| FVC at | | | | | | |
| week 52 | | | | | | |
| Adjusted | — | 93.1 | 78.0 | — | 71.8 | 58.9 |
| mean (95% | | (39.9, 146.3) | (24.2, 131.8) | | (24.9, 118.8) | (12.1, 105.8) |
| CI) | | | | | | |
| comparison | | | | | | |
| vs placebo | | | | | | |

| | P-value |
|---|---|
| Treatment-by-subgroup interaction 9 mg bid | 0.5555 |
| Treatment-by-subgroup interaction 18 mg bid | 0.5997 |

### 3.2.3 Adverse Events-Results of the FIBRONEER-ILD Trial for Progressive Pulmonary Fibrosis (PPF))

Table 7 shows that less serious adverse events and less fatal adverse events are observed under nerandomilast compared to placebo, whereas “any adverse events”, “severe adverse events” and “adverse events leading to discontinuation” were balanced.

TABLE 7
Fibroneer-ILD- adverse events summary

| | | Nera 9 | Nera 18 |
|---|---|---|---|
| | Placebo | mg bid | mg bid |
| | (n = 392) | (n = 393) | (n = 391) |
| Any adverse | 369 | 369 | 364 |
| event(s) | (94%) | (94%) | (93%) |
| Drug-related | 128 | 157 | 178 |
| adverse | (33%) | (40%) | (46%) |
| event(s)* | | | |
| Severe | 146 | 139 | 145 |
| adverse | (37%) | (35%) | (37%) |
| event(s) | | | |
| (CTCAE | | | |
| grade | | | |
| ≥3) | | | |
| Adverse | 46 | 39 | 44 |
| event(s) | (12%) | (10%) | (11%) |
| leading to | | | |
| permanent | | | |
| drug | | | |
| discontin- | | | |
| uation | | | |
| Serious | 168 | 150 | 154 |
| adverse | (43%) | (38%) | (39%) |
| event(s) | | | |
| Resulted in | 29 | 18 | 8 |
| death | (7%) | (5%) | (2%) |
| Was life | 20 | 22 | 16 |
| threatening | (5%) | (6%) | (4%) |
| Caused | 0 | 1 | 1 |
| disability or | (0%) | (<1%) | (<1%) |
| permanent | | | |
| damage | | | |
| Required or | 132 | 119 | 131 |
| prolonged | (34%) | (30%) | (34%) |
| hospitalization | | | |
| Other | 64 | 52 | 49 |
| medically | (16%) | (13%) | (13%) |
| important | | | |
| serious event | | | |

Table 8 shows that less fatal events under 9 mg bid. nerandomilast are only shown in the group of patients with antifibrotic background therapy, whereas under 18 mg bid. Nerandomilast less fatal events were observed for the groups with antifibrotic background therapy and without antifibrotic background therapy.

TABLE 8
Fibroneer-ILD: adverse events summary either without
background antifibrotic therapy or with background
antifibrotic therapy by nintedanib (for progressive
pulmonary fibrosis (PPF))

| | | | Nera | Nera |
|---|---|---|---|---|
| | | | 9 | 18 |
| | | | mg | mg |
| | | Placebo | bid | bid |
| Severe | Background | 61 | 61 | 77 |
| adverse | AF therapy | (36%) | (35%) | (45%) |
| event(s) | | | | |
| (CTCAE | | | | |
| grade | | | | |
| ≥3) | | | | |
| | No background | 85 | 78 | 68 |
| | AF therapy | (38%) | (35%) | (31%) |
| Adverse | Background | 18 | 17 | 23 |
| event(s) | AF therapy | (11%) | (10%) | (13%) |
| leading to | | | | |
| permanent | | | | |
| drug | | | | |
| discontin- | | | | |
| uation | | | | |
| | No background | 28 | 22 | 21 |
| | AF therapy | (13%) | (10%) | (10%) |
| Serious | Background | 70 | 65 | 74 |
| adverse | AF therapy | (41%) | (38%) | (43%) |
| event(s) | | | | |
| | No background | 98 | 85 | 80 |
| | AF therapy | (44%) | (39%) | (36%) |
| Fatal adverse | Background | 13 | 4 | 3 |
| event(s) | AF therapy | (8%) | (2%) | (2%) |
| | No background | 16 | 14 | 5 |
| | AF therapy | (7%) | (6%) | (2%) |

Table 9 shows that similarly like in the Fibroneer-IPF trial also in the Fibroneer-ILD trial diarrhoea is by far the most frequent adverse event. However, the frequencies are only slightly higher than in placebo and are comparable to the ones as observed in the Fibroneer-IPF trial. The safety and tolerability profile for both doses in patients with PPF are therefore favourable and similar to the established safety profile in patients with IPF.

TABLE 9
Fibroneer-ILD: most frequent adverse events (PT
frequency >5% in any group (for progressive
pulmonary fibrosis (PPF))

| | | Nera 9 | Nera 18 |
|---|---|---|---|
| | Placebo | mg bid | mg bid |
| | (n = 392) | (n = 393) | (n = 391) |
| Diarrhoea | 98 | 121 | 143 |
| | (25%) | (31%) | (37%) |
| Cough | 60 | 53 | 61 |
| | (15%) | (13%) | (16%) |
| Upper | 63 | 42 | 51 |
| respiratory | (16%) | (11%) | (13%) |
| tract infection | | | |
| COVID-19 | 63 | 44 | 47 |
| | (16%) | (11%) | (12%) |
| Condition | 65 | 52 | 38 |
| aggravated | (17%) | (13%) | (10%) |
| Nasopharyngitis | 45 | 49 | 43 |
| | (11%) | (12%) | (11%) |
| Depression | 47 | 41 | 43 |
| | (12%) | (10%) | (11%) |
| Pneumonia | 40 | 41 | 45 |
| | (10%) | (10%) | (12%) |
| Anxiety | 38 | 41 | 43 |
| | (10%) | (10%) | (11%) |
| Dyspnoea | 38 | 36 | 37 |
| | (10%) | (9%) | (9%) |
| Weight | 26 | 36 | 49 |
| decrease | (7%) | (9%) | (13%) |
| Nausea | 28 | 35 | 43 |
| | (7%) | (9%) | (11%) |

### CONCLUSION

Both, the results of the FIBRONEER-IPF phase III trial for the indication idiopathic pulmonary fibrosis (IPF) and of the FIBRONEER-ILD phase III trial for the indication progressive pulmonary fibrosis (PPF) report positive results in over a decade of clinical research in progressive fibrosing interstitial lung diseases (PF-ILD). Both of the investigated doses of nerandomilast in both phase III trials, 9 mg twice daily and 18 mg twice daily, were associated with a significant reduction in decline in FVC over 52 weeks, indicating a slowing of disease progression. Safety data for both doses and in both phase III trials suggested that the adverse events associated with nerandomilast are manageable.

### 4. PHASE I CLINICAL TRIAL ON THE BIOAVAILABILITY OF NERANDOMILAST IN THE PRESENCE OR ABSENCE OF ITRACONAZOLE (STRONG P450 3A (CYP3A) INHIBITOR)

### 4.1 Concept of the Trial

In another phase I study the relative bioavailability of a single oral dose of the PDE4B-inhibitor of formula III (nerandomilast) when administered alone or in combination with multiple oral doses of the strong competitive inhibitor for CYP3A itraconazole (strong P450 3A (CYP3A) inhibitor) has been determined in healthy men (healthy male volunteers). Itraconazole is an oral broad-spectrum imidazole antifungal agent and is a recommended probe drug to test in vivo inhibition of CYP3A. Based on in vitro data the PDE4B-inhibitor of formula III is mainly metabolized by CYP3A, a major drug-metabolizing enzyme.

This drug-drug interaction trial was an open-label, fixed sequence study with two treatment periods in 16 healthy male white participants with an median body mass index of 25.7 kg/m2 (2.8) and a median age of 42.3 years (10.2).

A single oral dose of 6 mg of nerandomilast was administered alone in the “reference treatment period”.

Once daily 200 mg itraconazole was administered for 12 days in the “test treatment period”. 6 mg of nerandomilast was administered on the fourth day of the “test treatment period”. The single dose treatments with nerandomilast in the reference treatment periods and the test treatment periods were separated by a “washout period” of at least 10 days (see FIG. 8). Blood samples were collected up to 119 h in the reference period and up to 215 h in the test period after the administration of nerandomilast to determine the nerandomilast plasma concentration in the absence and in the presence of itraconazole.

The primary endpoints of this study were AUC₀₋₁₁₉ of nerandomilast and the Cₘₐₓ of nerandomilast. Secondary endpoint was AUC_{0-∞} of nerandomilast. Further, the safety and tolerability of the drugs were also assessed based on the adverse events (AEs) reported.

### 4.2 Results of the Trial

Coadministrations of nerandomilast with 200 mg itraconazole delayed the peak plasma concentration of nerandomilast by approx. 1 h. The plasma concentration of nerandomilast decreased similarly in the terminal phase of both treatments (see FIG. 9).

When neradomilast was administered alone in the reference period, plasma concentrations of nerandomilast's major metabolite peaked 4 hours after the oral administration of 6 mg nerandomilast and was detectable up to 71 h post-dose in most individuals.

When nerandomilast was co-administered with 200 mg itraconazole, plasma concentrations of nerandomilast's major metabolite were below the quantifiable limit at all time points.

Further, co-administration of nerandomilast with 200 mg of the strong CYP3A-inhibitor itraconazole increased the AUC₀₋₁₁₉ by approx. 2.2-fold (see gMean ratio of test to reference treatment AUC₀₋₁₁₉=222.1%) and the Cₘₐₓ by approx. 1.3-fold (see gMean ratio of test to reference treatment Cₘₐₓ=128.0%).

TABLE 10
Pharmacokinetic parameters of nerandomilast after a single oral dose of 6 mg
nerandomilast alone or in combination with 200 mg itraconazole once daily:

| | | | | | 90% |
|---|---|---|---|---|---|
| | | | | gMean | confidence |
| | | | | ratio of | interval |
| | | Nerandomilast + | | test to | (CI) of |
| Nerandomilast | | Itraconazole | | reference | gMean |
| (N = 16) | | (N = 16) | | treatment | ratio |
| gMean | gSE | gMean | gSE | % | % |

Primary endpoints

| | | | | | | |
|---|---|---|---|---|---|---|
| AUC₁₋₁₁₉ in | 933 | 1.07 | 2073 | 1.07 | 222.1 | 203.5-242.5 |
| nmol/L | | | | | | |
| Cₘₐₓ in | 127 | 1.06 | 162 | 1.06 | 128.0 | 117.7-139.2 |
| nmol/L | | | | | | |

Secondary endpoint

| | | | | | | |
|---|---|---|---|---|---|---|
| AUC_{0-∞} | 952 | 1.08 | 2183 | 1.08 | 229.2 | 207.8-252.9 |
| in nmol h/L | | | | | | |

A less prominent increase in nerandomilast exposure is expectable when nerandomilast is co-administered with moderate or mild CYP3A-inhibitors.

4.3

### 4.4 Safety

A single dose of 6 mg of nerandomilast alone and in combination with multiple doses of 200 mg itraconazole was well tolerated by the healthy male participants in this trial.

TABLE 11
Adverse events (AEs) as reported in the trial

| | | | Nerando- | |
|---|---|---|---|---|
| | | | milast + | |
| | Nerando- | Itracon- | Itracon- | |
| | milast | azole | azole | Total |
| | (N = 16) | (N = 16) | (N = 16) | (N = 16) |
| Any AEs, | 5 | 5 | 9 | 11 |
| n (%) | (31.3) | (31.3) | (56.3) | (68.8) |
| Gastroin- | 2 | 3 | 5 | 9 |
| testinal | (12.5) | (18.8) | (31.3) | (56.3) |
| disorders | | | | |
| Diarrhea, | 2 | 1 | 5 | 7 |
| n (%) | (12.5) | (6.3) | (31.3) | (43.8) |
| Nausea, | — | 2 | — | 2 |
| n (%) | | (12.5) | | (12.5) |
| Nervous | 2 | — | 2 | 4 |
| system | (12.5) | | (12.5) | (25.0) |
| disorders | | | | |
| Headache, | 2 | — | 2 | 4 |
| n (%) | (12.5) | | (12.5) | (25.0) |

### 4.5 Conclusions

Co-administrations of nerandomilast with the strong CYP3A-inhibitor itraconazole increased the nerandomilast AUC₀₋₁₁₉ by 2.2-fold and Cₘₐₓ by 1.3-fold.

This means that the plasma concentrations of nerandomilast will be increased (more than doubled) in a patient that receives a co-treatment/co-administration of medicaments that are strong CYP3A-inhibitors.

Strong CYP3A-inhibitors are for example itraconazole, clarithromycin, telithromycin, nefazodone, ketoconazole, indinavir, lopinavir, nelfinavir, ritonavir, saquinavir, tipranavir, ceritinib, cobicistat, elvitegravir, idelalisib, paritaprevir, ombitasvir, dasabuvir, posaconazole, voriconazole, adagrasib, boceprevir, danoprevir, ensartinib, ensitrelvir, idelalisib, josamycin, LCL161, lonafarnib, mibefradil, mifepristone, morphothiadin, nirmatrelvir, paritaprevir, posaconazole, relacorilant, ribociclib, telaprevir, troleandomycin, tucatinib, vonoprazan and amoxicillin.

The above-mentioned results of the FIBRONEER-IPF and FIBRONEER-ILD phase III trials show clearly that IPF-patients and PPF-patients that received 9 mg twice daily of nerandomilast or that received 18 mg twice daily of nerandomilast showed not only an excellent treatment efficacy, but also an acceptable safety profile and good tolerability of nerandomilast in these doses and dose regimens. Consequently 18 mg nerandomilast administered twice daily is the highest dose/dose regimen that was tested in large numbers of IPF-patients and of PPF-patients for more than 52 weeks and was considered to be highly efficacious and safe with an acceptable tolerability. Higher doses/dose regimens of nerandomilast have not been tested regularly in larger numbers of humans or human IPF/PPF-patients over longer time periods and therefore should be precautiously avoided.

Since the above-mentioned itraconazole-trial showed that the trial participants that received nerandomilast in combination with the strong CYP3A-inhibitor itraconazole showed a 2.2-fold AUC₀₋₁₁₉ of nerandomilast and thereby showed a more than “doubled plasma concentration of nerandomilast” when the strong CYP3A-inhibitor itraconazole was co-administered, it can be concluded that patients suffering from a disease selected from IPF, PPF or SSc should only be treated twice daily with a pharmaceutical composition comprising 9 mg nerandomilast, when these patients are co-administered with a strong CYP3A-inhibitor.

In patients receiving 9 mg of nerandomilast twice daily who are concomitantly receiving a strong CYP3A-inhibitor the plasma concentration of nerandomilast will be doubled and correspond to the plasma concentration that is obtained by 18 mg twice daily nerandomilast dosing (in the absence of concomitant strong CYP3A-inhibitor use) which is according to the results of FIBRONEER-IPF and FIBRONEER-ILD considered to be safe and well tolerated. Nerandomilast dosing/dose regimens exceeding 9 mg of nerandomilast twice daily should not be applied to IPF-, PPF- or SSc-patients that receive concomitant treatment with a strong CYP3A-inhibitor taking into consideration the information so far received from the nerandomilast clinical trials.

### FIGURES

FIG. 1 shows the mean change from baseline in Forced Vital Capacity (FVC) (ml) at Week 52 of the Fibroneer-IPF phase III trial in the overall population (with or without antifibrotic background therapy).

FIG. 2 shows the mean change from baseline in Forced Vital Capacity (FVC) (ml) at Week 52 of the Fibroneer-IPF phase III trial in patients without antifibrotic background therapy.

FIG. 3 shows the mean change from baseline in Forced Vital Capacity (FVC) (ml) at Week 52 of the Fibroneer-IPF phase III trial in patients with nintedanib as antifibrotic background therapy.

FIG. 4 shows the mean change from baseline in Forced Vital Capacity (FVC) (ml) at Week 52 of the Fibroneer-IPF phase III trial in patients with pirfenidone as antifibrotic background therapy.

FIG. 5 shows the plasma concentration-time profiles of nerandomilast over 26 weeks by background antifibrotic therapy (bid=administered twice daily).

FIG. 6 shows the mean change from baseline in Forced Vital Capacity (FVC) (ml) at Week 52 of the Fibroneer-ILD phase III trial (for the indication PPF) in the overall population (with or without antifibrotic background therapy).

FIG. 7 shows the change from baseline in FVC (mL) over time in the Fibroneer-ILD phase III trial (for the indication PPF).

FIG. 8 shows the mean change from baseline in Forced Vital Capacity (FVC) (mL) at Week 52 of the Fibroneer-ILD phase III trial (for the indication PPF) in the patient population without antifibrotic background therapy (no background treatment with nintedanib).

FIG. 9 shows the mean change from baseline in Forced Vital Capacity (FVC) (mL) at Week 52 of the Fibroneer-ILD phase III trial (for the indication PPF) in the patient population with antifibrotic background therapy with nintedanib (patients taking nintedanib as background treatment).

FIG. 10 shows the single oral dose of 6 mg nerandomilast alone during the Reference Treatment and the 200 mg itraconazole administration once daily during the 12 days of the Test Treatment. 6 mg of nerandomilast was then administered on the fourth day of the Test Treatment. The nerandomilast single dose treatment in the reference and test treatment periods were separated by a washout period of at least 10 days. Blood samples were collected up to 119 h in the reference treatment period and up to 215 h in the test treatment period in the absence and in the presence of itraconazole.

FIG. 11 shows the gMean plasma-concentration—time profiles of nerandomilast after a single oral dose of 6 mg nerandomilast alone or in combination with 200 mg itraconazole once daily.

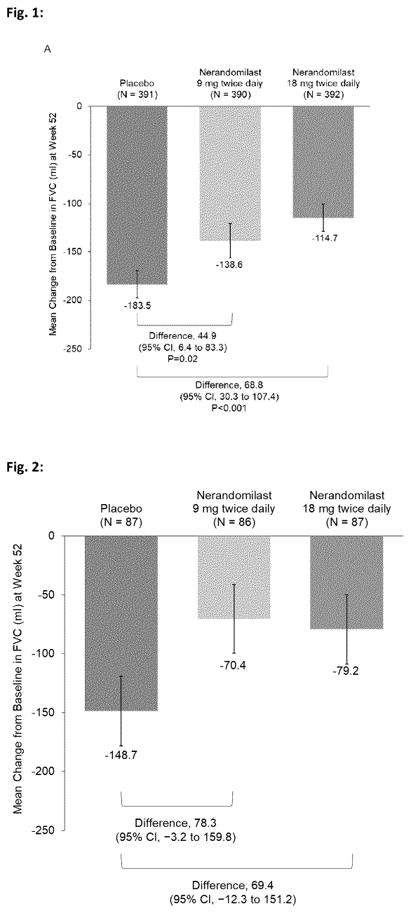

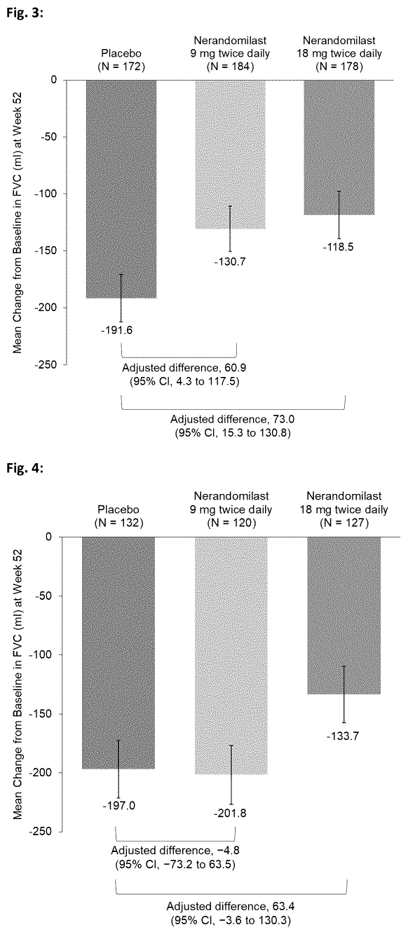

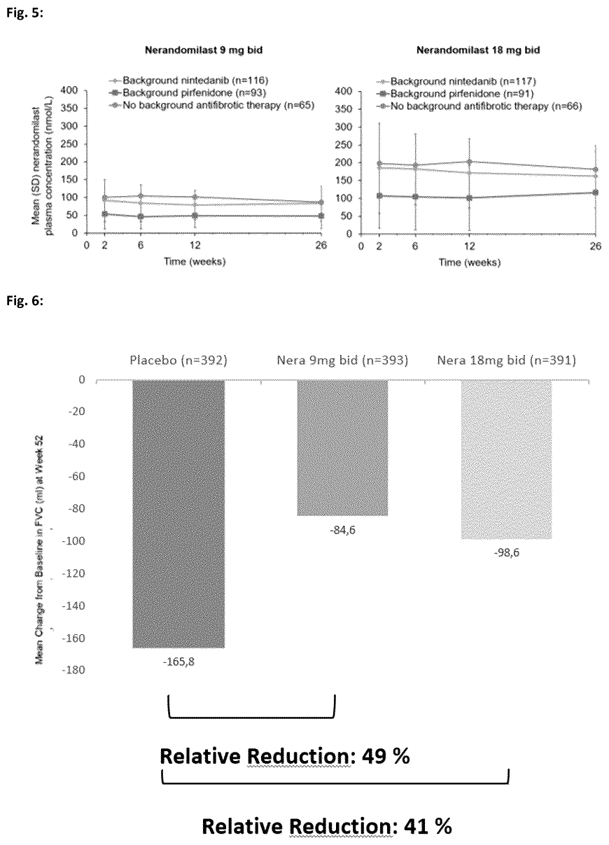

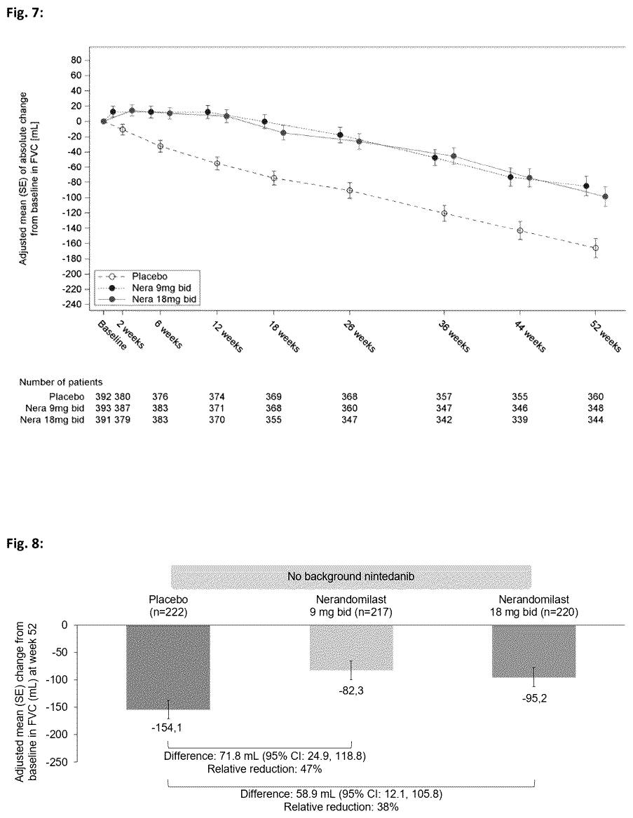

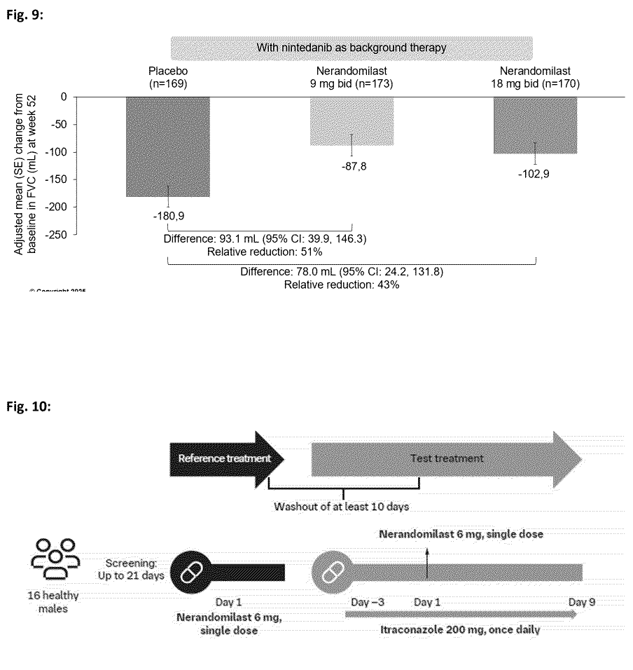

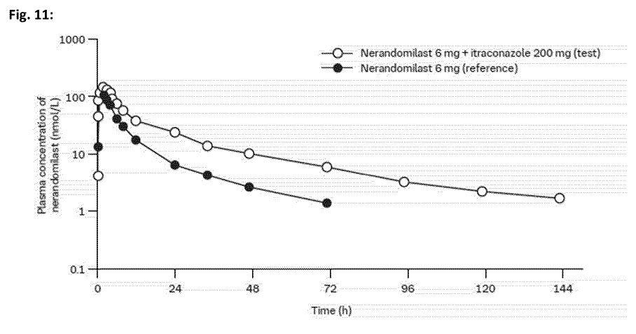

1. A method of treating a patient suffering from a disease selected from the group consisting of idiopathic pulmonary fibrosis (IPF), progressive pulmonary fibrosis (PPF) and systemic sclerosis (SSc), comprising the step of administering to said patient a pharmaceutical composition comprising 9 mg of the compound of formula III 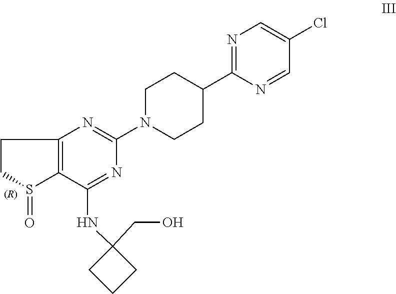
twice daily in combination with or concomitantly to a second pharmaceutical composition comprising a strong CYP3A-inhibitor (strong P450 3A (CYP3A) inhibitor).

2. The method of claim 1, wherein the strong CYP3A-inhibitor is selected from the group consisting of itraconazole, ketoconazole, clarithromycin, telithromycin, nefazodone, indinavir, lopinavir, nelfinavir, ritonavir, saquinavir and tipranavir, ceritinib, cobicistat, elvitegravir, idelalisib, paritaprevir, ombitasvir, dasabuvir, posaconazole, voriconazole, adagrasib, boceprevir, danoprevir, ensartinib, ensitrelvir, idelalisib, josamycin, LCL161, lonafarnib, mibefradil, mifepristone, morphothiadin, nirmatrelvir, paritaprevir, posaconazole, relacorilant, ribociclib, telaprevir, troleandomycin, tucatinib, vonoprazan and amoxicillin.

3. The method of claim 1, wherein the patient is suffering from idiopathic pulmonary fibrosis (IPF).

4. The method of claim 1, wherein the patient is suffering from progressive pulmonary fibrosis (PPF).

5. The method of claim 1, wherein the patient is suffering from systemic sclerosis (SSc).

6. The method of claim 2, wherein the patient is suffering from idiopathic pulmonary fibrosis (IPF).

7. The method of claim 2, wherein the patient is suffering from progressive pulmonary fibrosis (PPF).

8. The method of claim 2, wherein the patient is suffering from systemic sclerosis (SSc).
